(19) United States
(12) Patent Application Publication
(10) Pub. No.: US 2014/0235570 A1
(43) Pub. Date: Aug. 21, 2014

(54) THERAPEUTIC COMPOSITION FOR THE TREATMENT OF PERIANAL DISORDERS

(71) Applicant: David Jonathan Hochman, Winnipeg (CA)

(72) Inventor: David Jonathan Hochman, Winnipeg (CA)

(21) Appl. No.: 14/183,660

(22) Filed: Feb. 19, 2014

Related U.S. Application Data
(60) Provisional application No. 61/766,294, filed on Feb. 19, 2013.

(51) Int. Cl. A61K 31/7016; A61K 31/4422; A61K 9/00; A61K 31/167

(52) U.S. Cl. CPC A61K 31/7016; A61K 31/167; A61K 31/4422; A61K 9/0014; A61K 9/0031
USPC 514/53

(57) ABSTRACT

The invention relates to a composition for the treatment of perianal disorders. In particular, the composition includes three components: a) an analgesic, b) a cryoprotective agent and c) a muscle relaxant.

This application claims the benefit of priority of U.S. Provisional Patent Application No. 61/766,294, filed on Feb. 19, 2013, and which is incorporated herein in its entirety.

### FIELD OF THE INVENTION

The present invention relates to a composition for the treatment of perianal disorders. More specifically this invention relates to a topical composition comprising three active agents and the use of said composition in the treatment of conditions and disorders affecting the perianal region.

### BACKGROUND

Perianal disorders refers to disorders of the tissue located around the anus, the opening of the rectum to the outside of the body and the anal canal. Injury and trauma to the anal canal or surrounding tissue can result in extremely painful conditions which can be very difficult for individuals to tolerate and can be equally difficult to manage medically.

Examples of such perianal disorders include but are not limited to anal fissures, thrombosed external hemorrhoids and prolapsing internal hemorrhoids. These conditions are very painful and can be extremely difficult to heal. Additionally, managing the pain and wound healing from surgical procedures to this area, such as surgical hemorrhoidectomy, fissurectomy, lateral internal sphincterotomy, and fistulotomy, to name just a few, poses a challenge for both patients and the physicians treating them.

There have been numerous attempts to manage these painful conditions but none have been truly effective at managing this pain for a variety of reasons. For example, U.S. Pat. No. 5,196,405 teaches hemorrhoidal compositions containing disaccharide polysulfate-aluminum compounds such as sucralfate. Specifically, this patent teaches that the sucralfate can be used as a carrier, complexing with the wounded area and holding additional pharmaceutical compounds near the wounded area.

U.S. Pat. No. 6,395,736 and U.S. Pat. No. 6,627,632 teach compositions comprising NO donors and a number of other possible compounds, including an L-type Ca²⁺ channel blocker.

U.S. Pat. No. 6,242,010 teaches a composition comprising reduced glutathione and selenium as a selenoamino acid in a suitable carrier for topical applications for treating ano-rectal wounds.

The commercially available topical agents used to treat such disorders typically comprise, a topical steroid designed to reduce the local inflammation. However, these agents do nothing to directly treat the underlying pain, nor do they contribute to improving the individual's healing capacity. Additionally, if used too frequently, topical steroids can cause significant atrophy to the perianal skin that can predispose individuals to further injury and complications.

Topical and oral analgesics have also been used, and may temporarily help with the pain of perianal disorders, but fail to treat the underlying cause of the problem. These agents do not address the sphincter spasm, or hypertonicity and do little to improve blood supply to the area and to promote healing of the injured tissue.

Many topical agents have been tried to manage perianal disorders to mitigate the need for surgery or to aid in post-surgical recovery. Topical nitro-glycerine was once considered standard therapy as it reduces the anal sphincter tone and improves blood supply to the anal canal, but its significant side effects, including systemic hypotension and headaches, resulted in very poor patient compliance. With poor compliance, these disorders often went incompletely treated and resulted in unhealed lesions and continued pain for the individual.

Topical calcium channel blockers have largely replaced topical nitroglycerine in managing perianal disorders due to their reduced incidence of side-effects, especially headaches. Calcium channel blockers directly reduce the tone of the sphincter complex acting on the muscle fibers, again with the aim of improving blood supply to the injured perianal tissue, but, this drug too, is not perfect. Patients continue to experience pain even while using the medication as resolution of the pain is only achieved once the injured tissue has healed completely, often requiring up to 8 to 12 weeks. Topical calcium channel blockers, while successful for many patients, do not have success in all individuals and there are many patients who still experience chronic pain.

To date, there are few topical therapies that effectively treat pain from perianal disorders and most patients still require significant quantities of oral analgesics to manage their discomfort. Many of the oral analgesics are opioids, which can result in constipation and cause further discomfort when patients pass hard stools along the raw and healing surfaces of the injured tissue.

Surgical treatments have also been used to address these conditions. Surgery is typically reserved for patients who have failed more conservative therapies. The surgical division of a portion of the anal sphincter muscle is performed to reduce the basal sphincter tone and results in improved blood supply to the injured area, thereby allowing it to heal. But significant complications can result, as dividing this muscle is permanent and can potentially result in fecal incontinence and a significantly reduced quality of life. Furthermore, the surgery itself can result in significant pain during the healing process.

There remains a need for improved treatments for perianal disorders or conditions.

### SUMMARY

According to a one aspect, there is provided a composition for the treatment of perianal disorders comprising:
a) a first agent that is an analgesic;
b) a second agent that is a cytoprotective agent and
c) a third agent that is a muscle relaxant.

According to one embodiment, the analgesic is a sodium channel blocker, such as Lidocaine.

According to another embodiment, the cytoprotective agent is sucralfate.

According to a further embodiment, the muscle relaxant is a calcium channel blocker, such as nifedipine.

According to yet another embodiment, there is provided a composition comprising Lidocaine, nifedipine and sucralfate.

According to another aspect, there is provided a composition as described above, in the form of a pharmaceutical composition formulated for topical application.

According to a yet another aspect, there is provided a use of a composition as described above, for the treatment of perianal disorders.

According to a further aspect, there is provided a method of treating perianal disorders comprising administering to subject in need thereof a pharmaceutically effective amount of a composition as described above.

According to still a further aspect, there is provided a composition as described above, for use in the treatment of perianal disorders.

### DESCRIPTION OF THE PREFERRED EMBODIMENTS

Although any methods and materials similar or equivalent to those described herein can be used in the practice or testing of the present invention; examples of suitable methods and materials are now described. All publications mentioned herein are incorporated herein by reference.

Perianal disorders or conditions include any disorders or conditions associated with anal or rectal disease, injury or trauma, such as anal fissures, anal ulcers, or swollen and/or thrombosed hemorrhoids, and incision sites from surgical procedures.

Anal fissure, anal ulcer, and hemorrhoidal diseases are relatively common disorders. An anal fissure or ulcer is a tear or ulcer of the mucosa or lining tissue of the distal anal canal. An anal fissure or ulcer can be associated with another systemic or local disease. Typically, these disorders are confined to the anal mucosa. Symptoms associated with anal fissures or ulcers are anal pain and bleeding. The pain may be more pronounced during and after bowel movements.

Hemorrhoids are specialized vascular areas lying subjacent to the anal mucosa which assist in maintaining fecal continence. Pathological swelling and thrombosis of these vascular cushions manifests as prolapsing internal and thrombosed external hemorrhoids respectively, with resultant and associated pain and discomfort.

The pathophysiology of perianal pain from internal and external hemorrhoids, anal fissures and surgical procedures relates to direct trauma and disruption of the anal lining. This disruption exposes sensitive regional nerve fibers which directly causes pain but also results in secondary painful spasms of the anal sphincter complex.

The ability to heal lesions in this region of the anatomy relates to available blood supply to the injured area. As described, an injury to the anal canal lining and exposure of direct nerve-endings causes pain, and can result in a dramatic increase in the basal anal sphincter tone (hypertonicity). Hypertonicity, in addition to being extremely painful, also dramatically decreases capillary blood supply to the area, slowing down the body's ability to heal the injury. This process can result in a vicious cycle of pain, causing spasm which in turn causes more pain and also reduced blood supply to the area of injury, resulting in inability to heal the damaged tissue. This negative feedback loop can result in a long-standing or chronic condition which may take months or longer to heal.

While not wishing to be bound by theory, treatment failure of topical calcium channel blockers is thought to be secondary to the above described negative pain-spasm-reduced capillary blood flow feedback loop. Because treatment with a calcium channel blocker does not address pain, the pain still exists and the resultant high sphincter tone continues to reduce the blood supply to the area. The negative feed back loop may continue in spite of the administration of the calcium channel blocker. This reduced blood supply reduces the local absorption of the topical calcium channel blocker and without proper absorption of the agent, it cannot work as effectively and the injury persists. Additionally, because the calcium channel blocker does little to treat the immediate and ongoing pain related to the injury, the patient continues to suffer. Without relief from their discomfort, patient compliance with the therapy is poor and may lead to failed treatment.

It has been found that multifactorial treatment of perianal conditions can provide improved outcomes for patients. Namely, addressing the immediate and ongoing pain related to the disruption of the anal lining, combined with reducing the associated sphincter spasm, and improving the blood supply to the area to allow for healing, and improved absorption of medication has been found to offer both short and long term relief and to promote healing in patients afflicted with perianal disorders or conditions.

A composition comprising three agents has been prepared to address the three aspects of the negative feedback loop and act in a synergistic manner to provide immediate and sustained relief of pain relating to such lesions and to improve blood flow to promote healing.

The first agent is an analgesic such as a sodium-channel blocker, that provides immediate short-term pain relief. Local, topical application of the first agent immediately reduces pain, thereby increasing compliance. The reduction in pain also immediately reduces the associated pain-induced anal sphincter spasm which synergistically, further reduces the individual's pain. With decreased pain and a reduction of sphincter tone, blood supply to the site of injury begins to improve.

Although not acting directly on the sphincter complex itself, by providing immediate pain relief, the sodium channel blocker starts to break the negative feedback loop described above.

Examples of suitable sodium channel blockers include but are not limited to: Procaine, Benzocaine, Chloroprocaine, Cocaine, Cyclomethycaine, Dimethocaine/Larocaine, Piperocaine, Propoxycaine, Procaine/Novocaine, Proparacaine, Tetracaine/Amethocaine, Lidocaine, Articaine, Bupivacaine, Cinchocaine/Dibucaine, Etidocaine, Levobupivacaine, Lidocaine/Lignocaine, Mepivacaine, Prilocaine, Ropivacaine, Trimecaine/lidocaine. Topical lidocaine and bupivicaine have been found to be effective and easy to use.

The second agent is a cytoprotective agent. Sucralfate is a cytoprotective agent which is a binding agent and adheres to raw mucosal and epithelial surfaces. Sucralfate is capable of binding to proteins such as figrinogen to form insoluble complexes, which may act as a protective barrier. On topical application to the affected area, sucralfate reduces pain by covering the exposed and activated nerve endings in the anal canal at the site of injury, thereby providing sustained pain relief over a longer period of time than a sodium channel blocker. By binding to and covering these surfaces, there is less basal excitement of nerves to pain within the anal canal. By binding to these injured surfaces and protecting the nerve ending, pain is reduced resulting in less pain overall. Additionally, the protection provided by this agent protects the nerve endings so that there is significantly less pain during the acts of defecation and wiping where these surfaces would otherwise be directly exposed to repeated trauma.

The third agent of the composition is a muscle relaxant, such as a calcium channel blocker. Calcium channel blockers work by blocking voltage-gated calcium channels which decreases intracellular calcium which in turn leads to a reduction in muscle contraction.

Suitable calcium channel blockers may include but are not limited to be amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nilmodipine, nisoldipine, nitrendipine, pranidipine, verapamil and diltiazem.

As described above, the first and second agents of the composition act to reduce pain, allowing the anal sphincter complex to relax, thereby improving blood supply to the region. Improved blood flow allows for the third agent, a topical calcium channel blocker, to then be better absorbed into the tissues.

With improved absorption, the third agent, the calcium channel blocker, relaxes the sphincter complex even more effectively, directly reducing the basal muscle tone and directly improving the blood supply to the injured anal lining thereby promoting superior healing. The relaxed sphincter is by definition no longer in spasm, and results in the individual experiencing even less pain on a sustained basis.

It is this unique combination of agents which creates a favorable feedback loop that gives this combination of agents a unique effectiveness greater than would be expected.

Beginning with the topical sodium channel blocker, the immediate reduction in pain provides early patient relief but only for a limited time. However, this allows for a relatively pain-free period during which the sucralfate effectively binds to the ulcerated tissues and covers over exposed and irritated nerve fibers. This results in a more sustained reduction in basal pain and reduced pain during the acts of defecation and wiping.

By treating the anorectal pain through different mechanisms, these two agents favorably reduce the pain-induced spasm of the anal sphincter which then improves blood supply to the anal canal and allows for far more effective local absorption of the third agent, such as a topical calcium channel blocker. It is this agent which then achieves sustained relaxation of the anal sphincter and allows for sustained improvement in blood supply to the region and ultimately promotes tissue healing.

Additionally, by addressing the painful negative feedback loop directly, both the immediate pain relief provided by the sodium channel blocker and the more sustained pain reduction achieved with both the sucralfate and calcium channel blocker, promotes compliance in the patient resulting in regular application of the composition, which also leads to improved outcomes in the treatment of perianal disorders.

The sodium channel blocker as an analgesic alone, cannot heal perianal disorders.

Similarly, sucralfate as a binding-agent alone, fails to manage these disorders. Even calcium channel blockers which have been proven to be effective for the treatment of anal fissures and some other perianal conditions, cannot achieve success in refractory cases and even fails as primary therapy for some individuals. Only as a combination, can the negative feedback loop of pain-spasm-reduced capillary blood flow effectively be controlled, resulting in improved outcomes.

In one embodiment the composition comprises a topical sodium channel blocker in an amount from about 0.5 to about 5%, calcium channel blocker in an amount from about 0.1 to about 0.4%, and sucralfate in an amount from about 0.5 to about 10 grams per 50 mL. This composition has been found to achieve a success in managing such conditions beyond currently available therapies. In a further embodiment, the composition comprises a sodium channel blocker in an amount from about 1 to about 2% or about 10-20 mg/mL, a calcium channel blocker in an amount from about 0.2 to about 0.4% or about 2-4 mg/mL, and sucralfate of 4-6 grams per 50 mL. The balance of the compositions being one or more carrier agents.

In a further embodiment, the composition comprises lidocaine in an amount of 20 mg/mL of carrier, nifedipine in an amount of 2 mg/mL and sucralfate in an amount of 4 grams per 50 mL of carrier.

In a further aspect, the composition is formulated for topical application to skin. Topical formulations may include powders, sprays, ointments, pastes, creams, lotions, gels, solutions, patches, and liposomal preparations. In a further aspect the composition may be formulated as a suppository.

Formulation of the composition may further comprise one or more carriers or excipients suitable for topical application or suitable for formulation of a suppository. Examples of carriers or excipients include but are not limited to oil, including vegetable oils such as olive oil, sesame oil or nut oils, emulsions of water and oil, petrolatum, mineral oil, paraffins, microcrystalline wax, ceresine, wool fat, beeswax, macrogols 200, 300, 400, emulsifying wax, cetrimide, synthetic hydrocarbons, zinc oxide, alcohol, cellulose ethers, carbomer in water and water-alcohol mixtures, cocoa butter, polyethylene glycol, glycerin and gelatin.

In a particular embodiment the carrier is petroleum jelly.

In a further aspect the composition may include additives. The additives may be preservatives, buffers, propellants, colourants, fragrances, emulsifiers, and fat soluble anti-oxidant vitamins such as vitamins A, D, and E which can assist in wound repair and healing.

The invention will now be explained by way of examples. However, the examples are for illustrative purposes and the invention is not necessarily limited by the examples.

Patient 1

34-year-old patient with an anal fissure for 3 months, with symptoms of painful defecation and rectal bleeding on a daily basis, refractory to commercially available topical agents. Given the above three-agent compound applied three times per day (Lidocaine 2%, Nifedipine 0.2%, and Sucralfate 4 grams per 50 mL). Had resolution of pain upon first application and complete resolution of symptoms of rectal bleeding and painful defecation within 1 week of first application. Complete healing noted on exam 8 weeks following therapy.

Patient 2

57-year-old patient with a chronic anal fissure refractory to a topical calcium channel blocker alone. Given the above three-agent compound with resolution of symptoms within 2 weeks of initiating therapy (Lidocaine 2%, Nifedipine 0.2%, and Sucralfate 4 grams per 50 mL).

Patient 3

29-year-old patient with significant pain from thrombosed external hemorrhoids, associated with rectal bleeding. Given the above three-agent compound (Lidocaine 2%, Nifedipine 0.2%, and Sucralfate 4 grams per 50 mL), and had immediate pain relief upon first application with complete resolution of symptoms within 1 week. No oral opioids were required.

Patient 4

39-year-old patient who underwent a surgical hemorrhoidectomy with significant post-operative pain. Given the above three-agent compound (Lidocaine 2%, Nifedipine 0.2%, and Sucralfate 4 grams per 50 mL), with immediate improvement in pain relief and complete resolution of pain within 1 week. Oral opioid pain medication was required for only 2 days and only to a total dose of 30 mg of codeine per day.

Perianal pain from fissures, hemorrhoids, surgical interventions and other perianal pathology is multifactorial. Individual agents have not been found to be effective in alleviating the short and long term pain of these conditions and stimulate healing of the underlying cause of this pain.

This combination of a topical sodium channel blocker, binding agent, and calcium channel blocker is unique as it achieves excellent success in healing such disorders and providing effective pain relief. It is believed that the favorable feedback loop of reduced pain, resulting in less sphincter spasm, in turn resulting in improved regional blood flow allows for improved healing which is the hallmark of this new composition.

While the preferred embodiments of the invention have been described above, it will be recognized and understood that various modifications may be made therein, and the appended claims are intended to cover all such modifications which may fall within the spirit and scope of the invention.

1. A composition for the treatment of perianal disorders comprising:
a) a first agent that is an analgesic;
b) a second agent that is a cytoprotective agent and
c) a third agent that is a muscle relaxant.

2. The composition according to claim 1 wherein the first agent is a sodium channel blocker.

3. The composition according to claim 2 wherein the sodium channel blocker is procaine, benzocaine, chloroprocaine, cocaine, cyclomethycaine, dimethocaine/larocaine, piperocaine, propoxycaine, procaine/novocaine, proparacaine, tetracaine/amethocaine, lidocaine, articaine, bupivacaine, cinchocaine/dibucaine, etidocaine, levobupivacaine, lidocaine/lignocaine, mepivacaine, prilocaine, ropivacaine or trimecainelidocaine.

4. The composition according to claim 2 wherein the sodium channel blocker is lidocaine.

5. The composition according to claim 1 wherein the cytoprotective agent is sucralfate.

6. The composition according to claim 1 wherein the muscle relaxant is a calcium channel blocker.

7. The composition according to claim 1 wherein the calcium channel blocker is amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nilmodipine, nisoldipine, nitrendipine, pranidipine, verapamil or diltiazem.

8. The composition according to claim 6 wherein the calcium channel blocker is nifedipine.

9. The composition according to claim 1 comprising lidocaine, sucralfate and nifedipine.

10. The composition according to claim 1 wherein the lidocaine is 20 mg/mL of carrier.

11. The composition according to claim 1 wherein the nifedipine is 2 mg/mL.

12. The composition according to claim 1 wherein the sulcralfate is in the proportion of 4 grams per 50 mL.

13. A pharmaceutical composition comprising the composition of claim 1 and a pharmaceutically acceptable carrier.

14. A pharmaceutical composition according to claim 13 formulated for topical application.

15. A use of a composition according to claim 1 for the treatment of perianal disorders.

16. A method for treating perianal disorders comprising administering to subject in need thereof a pharmaceutically effective amount of a composition according to claim 14.

17. A composition according to claim 1 for use in the treatment of perianal disorders.

18. The method of according to claim 16 wherein the perianal disorder is anal fissure, hemorrhoids or a post-operative wound.

19. A pharmaceutical composition according to claim 13 formulated as a suppository.
